(19) United States
(12) Patent Application Publication
(10) Pub. No.: US 2014/0170768 A1
(43) Pub. Date: Jun. 19, 2014

(54) METHOD FOR MONITORING AND ASSESSING PITUITARY FUNCTION

(71) Applicant: Joel R. L. Ehrenkranz, Salt Lake City, UT (US)

(72) Inventor: Joel R. L. Ehrenkranz, Salt Lake City, UT (US)

(21) Appl. No.: 14/105,927

(22) Filed: Dec. 13, 2013

Related U.S. Application Data
(60) Provisional application No. 61/737,617, filed on Dec. 14, 2012.

(51) Int. Cl. G01N 33/50

(52) U.S. Cl. CPC G01N 33/5088
USPC 436/501

(57) ABSTRACT

Methods for monitoring pituitary function and for distinguishing Cushing's disease from Cushing's syndrome. The methods includes (1) providing a subject, (2) administering to the subject a daily dosage of a glucocorticoid antagonist, (3) monitoring adrenocorticotropic hormone (“ACTH”) and/or cortisol levels in the subject. Subjects having normal pituitary function typically show an increase in ACTH and/or cortisol levels following glucocorticoid antagonist therapy, while subjects having abnormal pituitary function will typically show no significant change in ACTH or cortisol following glucocorticoid antagonist therapy. Similarly, subjects having Cushing's disease show a significant rise in ACTH and cortisol levels following glucocorticoid antagonist therapy, while, in contrast, subjects having Cushing's syndrome show no significant change in ACTH and/or cortisol following glucocorticoid antagonist therapy.

### CROSS-REFERENCE TO RELATED APPLICATIONS

This application claims the benefit of and priority to U.S. Prov. Pat. App. Ser. No. 61/737,617 filed 14 Dec. 2012, the entirety of which is incorporated herein by reference.

### BACKGROUND

Glucocorticoids (“GC”) are a class of steroid hormones that bind to the glucocorticoid receptor (“GR”), which is present in almost every vertebrate cell. The name glucocorticoid (glucose+cortex) derives from their role in the regulation of the metabolism of glucose, their synthesis in the adrenal cortex, and their steroidal structure.

GCs are part of the feedback mechanism in the immune system that turns immune activity (inflammation) down. They are therefore used in medicine to treat diseases caused by an overactive immune system, such as allergies, asthma, autoimmune diseases and sepsis. They also interfere with some of the abnormal mechanisms in cancer cells, so they are used in high doses to treat cancer. This includes mainly inhibitory effects on lymphocyte proliferation (treatment of lymphomas and leukaemias) and mitigation of side effects of anticancer drugs.

GCs cause their effects by binding to the glucocorticoid receptor (GR). The activated GR complex, in turn, up-regulates the expression of anti-inflammatory proteins in the nucleus (a process known as transactivation) and represses the expression of pro-inflammatory proteins in the cytosol by preventing the translocation of other transcription factors from the cytosol into the nucleus (transrepression).

Cortisol (also commonly known as hydrocortisone) is a principle human glucocorticoid. It is essential for life, and it regulates or supports a variety of important cardiovascular, metabolic, immunologic, and homeostatic functions. Cortisol is shown below.

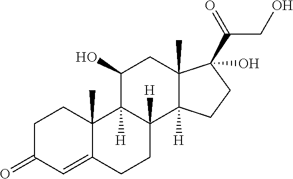

Cortisol is produced in the human body by the adrenal gland in the zona fasciculata. The release of cortisol is controlled by the hypothalamus and pituitary. The secretion of corticotropin-releasing hormone (“CRH”) by the hypothalamus triggers cells in the neighboring anterior pituitary to secrete another hormone, adrenocorticotropic hormone (“ACTH”), into the vascular system where it is carried by blood to the adrenal gland where it stimulates release of cortisol.

Cortisol's primary functions involve implementing a number of physiological mechanisms to optimize the body's ability to respond to a homeostatic threat. Specific effects of cortisol include increasing blood pressure, retaining sodium, increasing blood sugar through gluconeogenesis, suppressing the immune system, and modulating fat, protein and carbohydrate metabolism. Cortisol also decreases bone formation.

Cortisol is released in response to stress, sparing available glucose for the brain, generating new energy from stored reserves, and diverting energy away from low-priority activities (such as the immune system) in order to survive immediate threats or prepare for exertion.

However, prolonged cortisol secretion, which may be due to extended periods of stress or disease, can result in significant physiological damage. For example, prolonged elevation of cortisol levels is associated with hyperglycemia (i.e., elevated blood sugar), collagen loss throughout the body, inhibition of protein synthesis, elevated gastric acid secretion, immune system suppression, reduced bone formation, and alterations in mood and mentation. Long-term exposure to elevated levels of cortisol can lead to the development of osteoporosis, impaired learning, fertility problems, appetite and obesity, sleep difficulties and sleep deprivation, sexual dysfunction, and a host of other problems.

The primary control of cortisol levels in the blood is the pituitary gland hormone, ACTH. As a result, normal pituitary structure and function is essential for maintaining normal cortisol levels.

Two conditions caused by prolonged exposure to greatly elevated cortisol levels are Cushing's disease and Cushing's syndrome. Cushing's disease and Cushing's syndrome present with very similar symptoms and it is generally quite difficult to distinguish between them. Cushing's syndrome describes the signs and symptoms associated with prolonged exposure to inappropriately high levels of the hormone cortisol. This can be caused by taking glucocorticoid drugs, or diseases that result in excess cortisol, ACTH, or CRH levels. In contrast, Cushing's disease refers to a pituitary-dependent cause of Cushing's syndrome: a tumor (adenoma) in the pituitary gland produces large amounts of ACTH, causing the adrenal glands to produce elevated levels of cortisol.

Because of the overlap in the clinical presentation of Cushing's disease and Cushing's syndrome, they are difficult to distinguish from one another. When Cushing's disease or syndrome is suspected, a dexamethasone suppression test (i.e., administration of dexamethasone and frequent determination of cortisol and ACTH levels) is the test most commonly used to distinguish Cushing's disease from Cushing's syndrome. Dexamethasone is a synthetic glucocorticoid that mimics the effects of cortisol, including negative feedback on the pituitary gland. When dexamethasone is administered and a blood sample is tested, cortisol levels >50 nmol/L would be indicative of Cushing's syndrome because there is an ectopic source of cortisol or ACTH (such as adrenal adenoma) that is not inhibited by the dexamethasone. In contrast, in Cushing's disease, cortisol levels will normalize at least somewhat in response to dexamethasone treatment. It is also possible to distinguish between Cushing's disease and syndrome by performing similar tests with metyrapone and CRH.

However, such tests generally yield a certain number of false positives and false negatives. In addition, CRH-based testing is expensive and invasive as it usually involves sampling of blood from the petrosal sinuses, which is expensive and, in its own way, invasive. Accordingly, there exists in the art the need for better ways to monitor general pituitary function and, in particular, to distinguish between Cushing's disease from Cushing's syndrome.

### SUMMARY

Disclosed herein are methods for monitoring pituitary function and for distinguishing Cushing's disease from Cushing's syndrome. The methods includes (1) providing a subject, (2) administering to the subject a dosage of a glucocorticoid antagonist, (3) monitoring adrenocorticotropic hormone (“ACTH”) and cortisol levels in the subject. Subjects having normal pituitary function typically show an increase in ACTH and cortisol levels following glucocorticoid antagonist administration, while subjects having abnormal pituitary function will typically show no significant change in ACTH or cortisol following glucocorticoid antagonist administration. Subjects having Cushing's disease show a significant rise in ACTH and cortisol levels following glucocorticoid antagonist administration, while, in contrast, subjects having Cushing's syndrome show no significant change in ACTH or cortisol following glucocorticoid antagonist administration.

These and other objects and features of the present invention will become more fully apparent from the following description and appended claims, or may be learned by the practice of the invention as set forth hereinafter.

### DETAILED DESCRIPTION

Disclosed herein are methods for monitoring pituitary function and for distinguishing Cushing's disease from Cushing's syndrome. The methods includes (1) providing a subject, (2) administering to the subject a dosage of a glucocorticoid antagonist, (3) monitoring adrenocorticotropic hormone (“ACTH”) and cortisol levels in the subject. Subjects having normal pituitary function typically show an increase in ACTH and cortisol levels following glucocorticoid antagonist administration, while subjects having abnormal pituitary function will typically show no significant change in ACTH or cortisol following glucocorticoid antagonist administration. Similarly, subjects having Cushing's disease show a significant rise in ACTH and cortisol levels following glucocorticoid antagonist administration, while, in contrast, subjects having Cushing's syndrome show no significant change in ACTH or cortisol following glucocorticoid antagonist administration.

Hypercortisolism can result from excessive pituitary ACTH secretion, ectopic ACTH synthesis, overproduction of cortisol by an adrenal tumor, and rarely by extra-hypothalamic CRH production. Identifying the cause of excess cortisol production relies upon the sensitivity of pituitary-dependent ACTH secretion to inhibition by pharmacologic doses of dexamethasone. Mifepristone is a glucocorticoid receptor antagonist approved for the treatment of hyperglycemia in patients with Cushing's disease. It was hypothesized that mifepristone, by blocking glucocorticoid receptors, could be used to identify patients with pituitary-dependent hypercortisolism (i.e., Cushing's disease), as ACTH and cortisol levels should rise in these patients following mifepristone administration, while ACTH and cortisol should not change following mifepristone administration in patients with ectopic ACTH or primary adrenal disease (i.e., Cushing's syndrome).

Case One:

To test this hypothesis, patients with either Cushing's Disease or Cushing's Syndrome were administered mifepristone and monitored under the following conditions. Data are presented relating to ACTH and cortisol responses in patients with Cushing's disease following transphenoidal surgery (n=18) (i.e., surgery in which surgical instruments are inserted into part of the brain by going through the nose and the sphenoid bone (a butterfly-shaped bone at the base of the skull) in order to remove tumors of the pituitary gland), patients with Cushing's disease following transphenoidal surgery and pituitary radiation therapy (n=23), and patients with Cushing's syndrome secondary to ectopic ACTH secretion (n=4). All patients received mifepristone 300 mg orally per day. ACTH and cortisol were measured as close to 8 AM as possible using a centralized laboratory (Quest Diagnostics, Collegeville, Pa.) 14 days after commencing mifepristone therapy.

Patients with Cushing's disease who had not received radiation therapy (“no RT” in Table 1) had a significantly greater rise in ACTH and cortisol than patients with Cushing's disease who had received radiation therapy (“s/p RT” in Table 1). Patients with ectopic ACTH (i.e., those with Cushing's syndrome and not Cushing's disease) showed substantially no significant change in ACTH or cortisol following mifepristone therapy. These data are represented below in Table 1.

TABLE 1

| | | | | Statistical |
|---|---|---|---|---|
| | Day 0 | Day 14 | Fold change | significance |
| Cushing's Disease | | | | |
| ACTH (nmol/L) s/p RT | 46.1 ± 25.8 | 93.7 ± 71.6 | 1.40 ± .45 | p = .003 |
| ACTH no RT | 86.1 ± 67.7 | 106.2 ± 71.7 | 2.13 ± .96 | |
| cortisol (ng/L) s/p RT | 562.2 ± 164.9 | 874.3 ± 501.7 | 1.24 ± .23 | p = .02 |
| cortisol no RT | 618.9 ± 161.5 | 756.9 ± 227.8 | 1.56 ± .6 | |
| Cushing's Syndrome | | | | |
| due to ectopic ACTH | | | | |
| ACTH | 157.5 ± 140.3 | 200.0 ± 120.2 | 1.75 ± .75 | NS |
| cortisol | 1175.3 ± 395.2 | 1350.3 ± 277.8 | 1.21 ± .79 | NS |

These findings are consistent with the hypothesis that changes in ACTH and cortisol following mifepristone administration can be used to distinguish Cushing's disease after transphenoidal surgery from Cushing's disease after transphenoidal surgery and radiation therapy and from Cushing's syndrome due to ectopic ACTH secretion. A similar approach may be used to assess general pituitary function.

Case Two:

A 25 year old male with recurrent Cushing's disease following pituitary surgery received a dose of 300 mg mifepristone. ACTH and cortisol were measured at 8 AM prior to and at 8 AM the morning following the dose of mifepristone.

TABLE 2

| | pre-mifepristone | Post-mifepristone |
|---|---|---|
| ACTH | 84 picograms/ml plasma | 129 picograms/ml plasma |
| cortisol | 19.7 micrograms/deciliter | 32.9 micrograms/deciliter |

These results demonstrate the integrity of the pituitary-adrenal axis, as blocking the negative feedback of cortisol on the pituitary produced a rise in ACTH and cortisol. Likewise, these results demonstrate the principle that subjects having Cushing's disease show a significant rise in ACTH and cortisol levels following glucocorticoid antagonist administration.

Case Three:

A 76 year old woman was found to have adrenal insufficiency. ACTH and cortisol levels were low (ACTH<5 picograms/dl and cortisol 2.4 micrograms/deciliter following exogenous ACTH administration). Based upon the finding of both low ACTH and cortisol, the cause of her adrenal insufficiency could be due to pituitary or to hypothalamic disease. Her other pituitary hormones were normal and MRI of the pituitary did not show anatomic disease—i.e., she almost certainly did not have pituitary disease. ACTH and cortisol were measured at 8 AM on the day after she received a single dose of 300 mg mifepristone.

TABLE 3

| | pre-mifepristone | Post-mifepristone |
|---|---|---|
| ACTH | <5 picograms/ml plasma | 13 picograms/ml plasma |
| cortisol | 2.1 micrograms/deciliter | 11.3 micrograms/deciliter |

As stated above, subjects having normal pituitary function typically show an increase in ACTH and cortisol levels following glucocorticoid antagonist administration, while subjects having abnormal pituitary function will typically show no significant change in ACTH or cortisol following glucocorticoid antagonist administration. These results confirm intact pituitary function and show that hypothalamic regulation of pituitary ACTH release was defective and caused her adrenal insufficiency. This patient had been taking large doses of opiates daily for years. Opiates are known to interfere with the brain's regulation of pituitary ACTH secretion.

Accordingly, in an embodiment, a method for diagnosing pituitary function is disclosed. The method in includes (1) providing a subject (e.g., a human or an animal), (2) administering to the subject a daily dosage of a glucocorticoid antagonist, and (3) monitoring adrenocorticotropic hormone (“ACTH”) and cortisol levels in the subject.

Surprisingly and unexpectedly, it has been found that glucocorticoid antagonists have a differential effect on cortisol and/or ACTH levels in subjects depending on the state of the pituitary. This differential response can be used to diagnose pituitary disease and often to determine the source of the pituitary dysfunction, if any. This is superior to the current state of the art—e.g., the dexamethasone suppression test or CRH testing with petrosal sinus sampling, which can yield a certain number of false positives and false negatives.

In subjects having normal pituitary function, an increase in ACTH and cortisol levels following glucocorticoid antagonist therapy will typically be observed. That is, an intact and functional pituitary secretes increased amounts of ACTH when the negative feedback of cortisol on pituitary ACTH secretion is removed or blocked, such as by a glucocorticoid receptor antagonist. An intact and normally functioning pituitary will respond to this block in negative feedback by increasing the secretion of ACTH, which in turn leads to increased cortisol secretion.

In contrast, subjects having abnormal pituitary function will typically show no significant change in ACTH or cortisol following glucocorticoid antagonist therapy. That is, a pituitary that is not functioning normally will not increase ACTH secretion in response to a block in negative feedback.

Thus, blocking the negative feedback of cortisol on pituitary secretion of ACTH with a glucocorticoid antagonist can be used to demonstrate whether or not one component of pituitary function is intact. If the pituitary is intact, ACTH and cortisol levels will rise following blockade of the ACTH-secreting anterior pituitary cells' glucocorticoid receptors. If the pituitary is not working, blocking the pituitary's glucocorticoid receptors will not produce increased levels of ACTH and cortisol. Thus, looking at changes in ACTH and cortisol following the administration of glucocorticoid receptor antagonist represents a method to assess normal pituitary function.

In another embodiment, a method for distinguishing Cushing's disease from Cushing's syndrome is described. The method includes (1) providing a subject having one of Cushing's disease or Cushing's syndrome, (2) administering to the subject a daily dosage of a glucocorticoid antagonist, (3) monitoring adrenocorticotropic hormone (“ACTH”) and cortisol levels in the subject.

Surprisingly and unexpectedly, it has been found that even though Cushing's disease and Cushing's syndrome present with almost identical symptoms glucocorticoid antagonists have a differential effect on cortisol and/or ACTH levels in subjects depending on whether they have Cushing's disease or Cushing's syndrome. This differential response can be used to distinguish between Cushing's disease and Cushing's syndrome and often to determine the source of the disease. Typically, subjects having Cushing's disease show a significant rise in ACTH and cortisol levels following glucocorticoid antagonist therapy. In contrast, subjects having Cushing's syndrome show no significant change in ACTH or cortisol following glucocorticoid antagonist therapy. As discussed above, this is superior to the current state of the art—e.g., the dexamethasone suppression test.

In addition to the above described cases with respect to pituitary dysfunction, Cushing's disease, and Cushing's syndrome, differential response to glucocorticoid antagonists can also be used to discriminate between a number of additional disease states. For example, in a patient with Addison's disease or adrenal insufficiency, if ACTH and/or cortisol do not rise in response to a glucocorticoid antagonist it tells you that the patient has pituitary disease. A diagnosis of pituitary disease is typically followed by additional blood tests of pituitary function (e.g., luteinizing hormone (LH), follicular stimulation hormone (FSH), thyroid stimulation hormone (TSH), free T4, prolactin, etc.) and pituitary imaging (e.g., MRI or CT). The patient also needs to be treated for panhypopituitarism. Panhypopituitarism may, for example, be treated by administering the products of the effector glands: hydrocortisone (cortisol) for adrenal insufficiency, levothyroxine for hypothyroidism, testosterone for male hypogonadism, and estradiol for female hypogonadism (usually with a progestogen to inhibit unwanted effects on the uterus). Growth hormone is available in synthetic form, but needs to be administered parenterally (by injection). Antidiuretic hormone can be replaced by desmopressin (DDAVP) tablets or nose spray. If the patient's ACTH and/or cortisol do rise in response to a glucocorticoid antagonist, it means that their pituitary function is intact and that deficiencies in thyroid, adrenal, gonadal or renal function are the result of defects in the brain's regulation of the pituitary gland. Such a case of a problem with the brain's regulation of the pituitary was described above in Case Three.

If the patient has excess cortisol, i.e., Cushing's syndrome, and the ACTH and/or cortisol do not rise in response to a glucocorticoid antagonist, it tells you that the patient's excess ACTH and/or cortisol is coming from the adrenal gland or ectopic manufacture of ACTH (usually seen with malignancies). If the patient's ACTH and/or cortisol do rise following glucocorticoid administration it tells you that they have Cushing's disease (the pituitary is making too much ACTH) or that their excess cortisol is functional (e.g. due to depression or alcoholism). Such patients need further evaluation for Cushing's disease and treatment for excess cortisol.

Occasionally diabetes mellitus, hypertension, osteoporosis, electrolyte abnormalities, obesity, depression, easy bruising, muscle weakness, and the like can be due to excess cortisol. The methods described herein may be used to identify whether physiologic control of cortisol secretion is present. Likewise, the methods described herein may be useful in identifying whether excess cortisol production is present, and what the source of the excess control production might be.

In one embodiment, changes in ACTH and cortisol levels, if present, can be observed in the subject within one (1) day following glucocorticoid antagonist therapy. In another embodiment, changes in ACTH and cortisol levels, if present, can be observed in the subject within 1-14 days following glucocorticoid antagonist therapy.

Numerous examples of steroidal and non-steroidal glucocorticoid receptor antagonists are known in the art. Examples steroidal and non-steroidal glucocorticoid receptor antagonists can be found, for example, in U.S. Pat. Pub. Nos. 2010/0261693, 2012/0238549, 2012/0225876, 2012/0225856, and 2012/0220565 and U.S. Pat. Nos. 8,143,280 and 8,299,123, the entireties of which are incorporated herein by reference.

Examples of steroidal glucocorticoid receptor antagonists include, but are not limited to, mifepristone, monodemethylated mifepristone, didemethylated mifepristone, 17-α-[3′-hydroxy-propynyl]mifepristone, ulipristal (CDB-2914), CDB-3877, CDB-3963, CDB-3236, CDB-4183, cortexolone, dexamethasone-oxetanone, 19-nordeoxycorticosterone, 19-norprogesterone, cortisol-21-mesylate, dexamethasone-21-mesylate, 11(-(4-dimethylaminoethoxyphenyl)-17(-propynyl-17(-hydroxy-4,9-estradien—3one, and 17(-hydroxy-17(-19-(4-methylphenyl)androsta-4,9(11)-dien-3-one, and combinations thereof.

Examples of non-steroidal glucocorticoid receptor antagonists include, but are not limited to N-(2-[4,4′,441-trichlorotrityl]oxyethyl)morpholine; 1-(2[4,4′,4″-trichlorotrityl]oxyethyl)-4-(2-hydroxyethyl)piperazine dimaleate; N-([4,4′,4″]-trichlorotrityl)imidazole; 9-(3-mercapto-1,2,4-triazolyl)-9-phenyl-2,7-difluorofluorenone; 1-(2-chlorotrityl)-3,5-dimethylpyrazole; 4-(morpholinomethyl)-A-(2-pyridyl)benzhydrol; 5-(5-methoxy-2-(N-methylcarbamoyl)-phenyl)dibenzosuberol; N-(2-chlorotrityl)-L-prolinol acetate; 1-(2-chlorotrityl)-1,2,4-triazole; 1,S-bis(4,4′,4″-trichlorotrityl)-1,2,4-triazole-3-thiol; 4.alpha.(S)-Benzyl-2(R)-chloroethynyl-1,2,3,4,4.alpha., 9,10,10.alpha.(R)—octahydro-phenanthrene-2,7-diol (“CP 394531”), 4.alpha.(S)-Benzyl-2(R)-prop-1-ynyl-1,2,3,4,4.alpha., 9,10,10.alpha.(R)-oc-tahydro-phenanthrene-2,7-diol (“CP-409069”), trans-(1R,2R)-3,4-dichloro-N-methyl-N-[2-1 pyrrolidinyl)cyclohexyl]benzeneacetamide, bremazocine, ethylketocyclazocine, naloxone compounds of Formula I

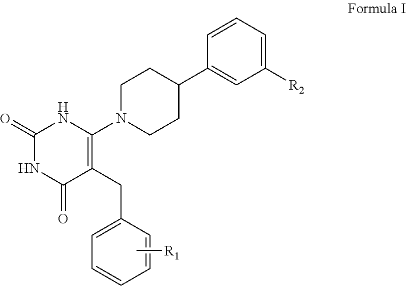

wherein R1 is H and R2 is H or Cl, or R1 is o-chloro or m-chloro and R2 is H, or compounds of Formula II

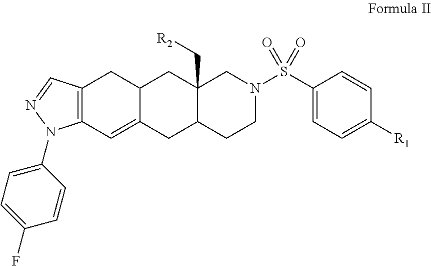

wherein R1 is F and R2 is pyrrolidine, or R1 is t-butyl and R2 is selected from the group consisting of H, a phenyl group, and —CH₂—O—CH₃.

In one embodiment, the glucocorticoid antagonist administered to the subject comprises at least one of mifepristone, onapristone, or Cissus quadrangularis. In another embodiment, the Cissus quadrangularis comprises a chemical extract of Cissus quadrangularis plant material.

In one embodiment, the glucocorticoid antagonist includes a daily dose mifepristone administered for at least 1 day. In one embodiment, the daily dose mifepristone ranges from about 100 mg/day to about 2000 mg/day, or about 300 mg/day.

In one embodiment the monitoring includes monitoring in an outpatient setting. This is superior to dexamethisone, metyrapone, CRH, testing or urinary cortisol testing.

In one embodiment, cortisol and/or ACTH levels in a subject may be assayed by testing one or more body fluids of the subject to determine if cortisol and/or ACTH levels change in response to administration of a glucocorticoid antagonist (as compared to a baseline control). For example, cortisol level can be determined by assaying the blood or the saliva or the urine of the subject.

In an embodiment, a lateral flow assay device that can be used to assay cortisol and/or ACTH levels is described. The device includes a base, an absorbent test strip for analyzing an analyte of interest in an experimental sample positioned above the base, and an absorbent calibration strip for running at least one calibration standard positioned above the base in proximity to the absorbent test strip. The device further includes a first sample application zone positioned between a distal end and a proximal end the first absorbent strip, and a second sample application zone positioned between a distal end and a proximal end the second absorbent strip. A volume of a liquid test sample applied to the first sample application zone and a volume of a liquid calibration standard applied to the second sample application zone each diffuse through their respective absorbent strips from the distal end to the proximal end. Accordingly, the analyte of interest, if present in the experimental sample, and the calibration standard interact with at least a first reporter (e.g., an antibody) selected to interact with the analyte of interest and immobilized on the first and second absorbent strips to yield a detectable signal.

Further discussion of such lateral flow assay devices can be found in U.S. Prov. Pat. App. Ser. No. 61/533,959 filed 13 Sep. 2011, U.S. patent application Ser. No. 13/612,293 filed 12 Sep. 2012, U.S. patent application Ser. No. 14/070,276 filed 1 Nov. 2013, U.S. Prov. App. Ser. No. 61/740,975 filed 21 Dec. 2012, U.S. Prov. Pat. App. Ser. No. 61/625,368 filed 17 Apr. 2012, U.S. patent application Ser. No. 13/862,176 filed 12 Apr. 2013, U.S. Prov. Pat. App. Ser. No. 61/625,390 filed 17 Apr. 2012, and U.S. patent application Ser. No. 13/862,184 filed 12 Apr. 2013, the entireties of which are incorporated herein by reference. In addition, discussion of electro-optical devices that can be used, for example, to read, quantify, and report the results provided by such lateral flow assay devices, which may be used in one or more of the methods described herein, may be found in U.S. Prov. Pat. App. Ser. No. 61/533,959 filed 13 Sep. 2011, U.S. patent application Ser. No. 13/612,293 filed 12 Sep. 2012, U.S. patent application Ser. No. 14/070,276 filed 1 Nov. 2013, U.S. Prov. App. Ser. No. 61/740,975 filed 21 Dec. 2012, U.S. Prov. Pat. App. Ser. No. 61/625,368 filed 17 Apr. 2012, U.S. patent application Ser. No. 13/862,176 filed 12 Apr. 2013, U.S. Prov. Pat. App. Ser. No. 61/625,390 filed 17 Apr. 2012, and U.S. patent application Ser. No. 13/862,184 filed 12 Apr. 2013.

In a specific, non-limiting example of the methods described herein, the methods may further include monitoring salivary cortisol in the subject. In one embodiment, salivary cortisol levels can be measured by assaying a saliva sample with a lateral flow immunoassay device like those described above.

In one embodiment, salivary cortisol can be assayed by applying a saliva sample to the test sample pad and a calibration sample to the calibration sample pad and running the samples out on the lateral flow device. The results of such a lateral flow assay can be read and quantified with an electro-optical device that is configured for reading such an assay.

In one embodiment, the results of an assay for cortisol and/or ACTH levels may be interpreted with the assistance of a software algorithm—e.g., a so-called decision support algorithm. For example, based on whether or not cortisol and/or ACTH levels rise in response to administration of a glucocorticoid receptor antagonist, the software algorithm may be programmed to assist a practitioner in determining whether or not a subject has a normally functioning pituitary (if the pituitary is intact, ACTH and cortisol levels will generally rise following administration of a glucocorticoid receptor antagonist; if the pituitary is not working, blocking the pituitary's glucocorticoid receptors will not produce increased levels of ACTH and cortisol). Likewise, based on whether or not cortisol and/or ACTH levels rise in response to administration of a glucocorticoid receptor antagonist, the software algorithm may be programmed to assist a practitioner in determining whether or not a subject has one or more pituitary disorders, such as, but not limited to, Cushing's syndrome or Cushing's disease (with or without ectopic ACTH). Typically, subjects having Cushing's disease show a significant rise in ACTH and cortisol levels following glucocorticoid antagonist therapy. In contrast, subjects having Cushing's syndrome show no significant change in ACTH or cortisol following glucocorticoid antagonist therapy.

In one embodiment, the software algorithm may be programmed to aid in determining whether or not any observed changes in cortisol and/or ACTH are significant. Likewise, the software algorithm may be programmed to use the ACTH and/or cortisol response to a glucocorticoid antagonist to evaluate pituitary function, identify the cause of Cushing's syndrome, and rule out hypercortisolism in patients observed with one or more of diabetes mellitus, hypertension, osteoporosis, electrolyte abnormalities, obesity, depression, easy bruising, or muscle weakness.

In one embodiment, the software algorithm may exist in an electronic form accessible by the electro-optical device described above. The software algorithm accessible by the electro-optical device may be programmed for one or more of converting a response from an assay for cortisol and/or ACTH to a numerical value for a concentration of cortisol and/or ACTH, communicating with one or more remote computer or cellphone networks for data upload of cortisol and/or ACTH concentration values to a medical records database, querying a data analysis algorithm for, for example, determining whether or not an observed change in cortisol and/or ACTH levels is significant, evaluating pituitary function, identifying the cause of Cushing's syndrome, querying a decision support algorithm, and the like. Additional discussion of such software algorithms may be found in U.S. Prov. Pat. App. Ser. No. 61/533,959 filed 13 Sep. 2011, U.S. patent application Ser. No. 13/612,293 filed 12 Sep. 2012, U.S. patent application Ser. No. 14/070,276 filed 1 Nov. 2013, U.S. Prov. App. Ser. No. 61/740,975 filed 21 Dec. 2012, U.S. Prov. Pat. App. Ser. No. 61/625,368 filed 17 Apr. 2012, U.S. patent application Ser. No. 13/862,176 filed 12 Apr. 2013, U.S. Prov. Pat. App. Ser. No. 61/625,390 filed 17 Apr. 2012, and U.S. patent application Ser. No. 13/862,184 filed 12 Apr. 2013.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A method for assessing pituitary function, comprising:
providing a subject;
administering to the subject a dosage of a glucocorticoid antagonist; and
monitoring adrenocorticotropic hormone (“ACTH”) and/or cortisol levels in the subject,
wherein subjects having normal pituitary function show an increase in ACTH and/or cortisol levels following glucocorticoid antagonist therapy, and wherein subjects having abnormal pituitary function show substantially no significant change in ACTH and/or cortisol following glucocorticoid antagonist therapy.

2. The method of claim 1, wherein changes in ACTH and/or cortisol levels can be observed in the subject within one (1) day following glucocorticoid antagonist therapy.

3. The method of claim 1, wherein changes in ACTH and/or cortisol levels can be observed in the subject within 1-14 days following glucocorticoid antagonist therapy.

4. The method of claim 1, wherein the glucocorticoid antagonist comprises a steroidal glucocorticoid receptor antagonist selected from the group consisting of mifepristone, monodemethylated mifepristone, didemethylated mifepristone, 17-α-[3′-hydroxy-propynyl]mifepristone, ulipristal (CDB-2914), CDB-3877, CDB-3963, CDB-3236, CDB-4183, cortexolone, dexamethasone-oxetanone, 19-nordeoxycorticosterone, 19-norprogesterone, cortisol-21-mesylate, dexamethasone-21-mesylate, 11(-(4-dimethylaminoethoxyphenyl)-17(-propynyl-17(-hydroxy-4,9-estradien—3one, and 17(-hydroxy-17(-19-(4-methylphenyl)androsta-4,9(11)-dien-3-one, and combinations thereof.

5. The method of claim 1, wherein the glucocorticoid antagonist comprises a non-steroidal glucocorticoid receptor antagonist selected from the group consisting of N-(2-[4,4′,441-trichlorotrityl]oxyethyl)morpholine; 1-(2[4,4′,4″-trichlorotrityl]oxyethyl)-4-(2-hydroxyethyl)piperazine dimaleate; N-([4,4′,4″]-trichlorotrityl)imidazole; 9-(3-mercapto-1,2,4-triazolyl)-9-phenyl-2,7-difluorofluorenone; 1-(2-chlorotrityl)-3,5-dimethylpyrazole; 4-(morpholinomethyl)-A-(2-pyridyl)benzhydrol; 5-(5-methoxy-2-(N-methylcarbamoyl)-phenyl)dibenzosuberol; N-(2-chlorotrityl)-L-prolinol acetate; 1-(2-chlorotrityl)-1,2,4-triazole; 1,S-bis(4,4′,4″-trichlorotrityl)-1,2,4-triazole-3-thiol; 4.alpha.(S)-Benzyl-2(R)-chloroethynyl-1,2,3,4,4.alpha., 9,10,10.alpha.(R)—octahydro-phenanthrene-2,7-diol (“CP 394531”), 4.alpha.(S)-Benzyl-2(R)-prop-1-ynyl-1,2,3,4,4.alpha., 9,10,10.alpha.(R)-oc-tahydro-phenanthrene-2,7-diol (“CP-409069”), trans-(1R,2R)-3,4-dichloro-N-methyl-N-[2-1 pyrrolidinyl)cyclohexyl]benzeneacetamide, bremazocine, ethylketocyclazocine, naloxone compounds of Formula I 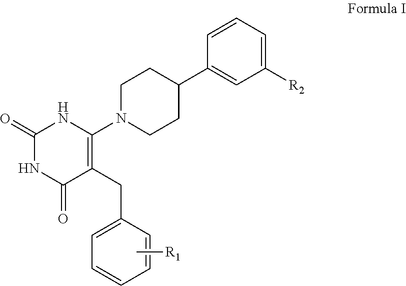
wherein R1 is H and R2 is H or Cl, or R1 is o-chloro or m-chloro and R2 is H, or compounds of Formula II 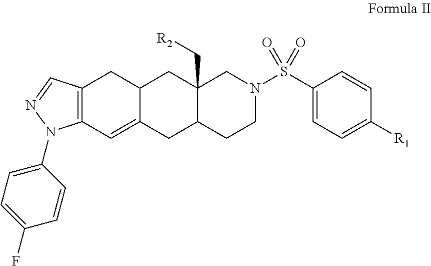
wherein R1 is F and R2 is pyrrolidine, or R1 is t-butyl and R2 is selected from the group consisting of H, a phenyl group, and —CH₂—O—CH₃.

6. The method of claim 1, wherein the glucocorticoid antagonist comprises at least one of mifepristone, onapristone, or Cissus quadrangularis.

7. The method of claim 6, wherein the Cissus quadrangularis comprises a chemical extract of Cissus quadrangularis plant material.

8. The method of claim 1, wherein the glucocorticoid antagonist comprises a daily dose mifepristone administered for at least 1 day.

9. The method of claim 1, wherein the monitoring includes monitoring in an outpatient setting.

10. A method for distinguishing Cushing's Disease from Cushing's Syndrome, comprising:
providing a subject having one of Cushing's Disease or Cushing's Syndrome;
administering to the subject a daily dosage of a glucocorticoid antagonist; and
monitoring adrenocorticotropic hormone (“ACTH”) and/or cortisol levels in the subject,
wherein subjects having Cushing's disease show a significant rise in ACTH and/or cortisol levels following glucocorticoid antagonist therapy and wherein subjects having Cushing's syndrome show no significant change in ACTH or cortisol following glucocorticoid antagonist therapy.

11. The method of claim 10, wherein changes in ACTH and/or cortisol levels can be observed in a subject having Cushing's disease within one (1) day following glucocorticoid antagonist therapy.

12. The method of claim 10, wherein changes in ACTH and/or cortisol levels can be observed in a subject having Cushing's disease within 1-14 days following glucocorticoid antagonist therapy.

13. The method of claim 1, wherein the glucocorticoid antagonist comprises a steroidal glucocorticoid receptor antagonist selected from the group consisting of mifepristone, monodemethylated mifepristone, didemethylated mifepristone, 17-α-[3′-hydroxy-propynyl]mifepristone, ulipristal (CDB-2914), CDB-3877, CDB-3963, CDB-3236, CDB-4183, cortexolone, dexamethasone-oxetanone, 19-nordeoxycorticosterone, 19-norprogesterone, cortisol-21-mesylate, dexamethasone-21-mesylate, 11 (-(4-dimethylaminoethoxyphenyl)-17(-propynyl-17(-hydroxy-4,9-estradien—3one, and 17(-hydroxy-17(-19-(4-methylphenyl)androsta-4,9(11)-dien-3-one, and combinations thereof.

14. The method of claim 1, wherein the glucocorticoid antagonist comprises a non-steroidal glucocorticoid receptor antagonist selected from the group consisting of N-(2-[4,4′,441-trichlorotrityl]oxyethyl)morpholine; 1-(2[4,4′,4″-trichlorotrityl]oxyethyl)-4-(2-hydroxyethyl)piperazine dimaleate; N-([4,4′,4″]-trichlorotrityl)imidazole; 9-(3-mercapto-1,2,4-triazolyl)-9-phenyl-2,7-difluorofluorenone; 1-(2-chlorotrityl)-3,5-dimethylpyrazole; 4-(morpholinomethyl)-A-(2-pyridyl)benzhydrol; 5-(5-methoxy-2-(N-methylcarbamoyl)-phenyl)dibenzosuberol; N-(2-chlorotrityl)-L-prolinol acetate; 1-(2-chlorotrityl)-1,2,4-triazole; 1,S-bis(4,4′,4″-trichlorotrityl)-1,2,4-triazole-3-thiol; 4.alpha.(S)-Benzyl-2(R)-chloroethynyl-1,2,3,4,4.alpha., 9,10,10.alpha.(R)—octahydro-phenanthrene-2,7-diol (“CP 394531”), 4.alpha.(S)-Benzyl-2(R)-prop-1-ynyl-1,2,3,4,4.alpha.,9,10,10.alpha.(R)-oc-tahydro-phenanthrene-2,7-diol (“CP-409069”), trans-(1R,2R)-3,4-dichloro-N-methyl-N-[2-1 pyrrolidinyl)cyclohexyl]benzeneacetamide, bremazocine, ethylketocyclazocine, naloxone compounds of formula I 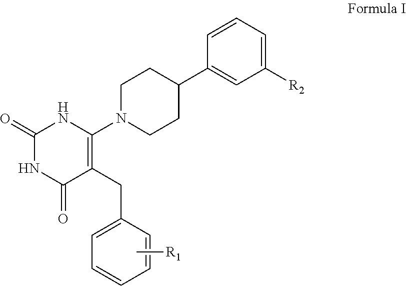
wherein R1 is H and R2 is H or Cl, or R1 is o-chloro or m-chloro and R2 is H, or compounds of formula II 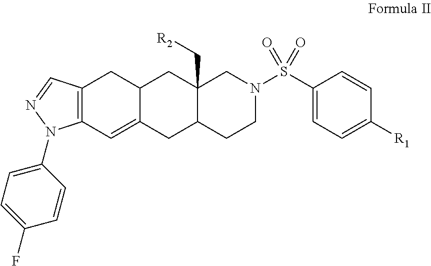
wherein R1 is F and R2 is pyrrolidine, or R1 is t-butyl and R2 is selected from the group consisting of H, a phenyl group, and —CH₂—O—CH₃.

15. The method of claim 10, wherein the glucocorticoid antagonist comprises at least one of mifepristone, onapristone, or Cissus quadrangularis.

16. The method of claim 15, wherein the Cissus quadrangularis comprises a chemical extract of Cissus quadrangularis plant material.

17. The method of claim 10, wherein the glucocorticoid antagonist comprises a daily dose mifepristone administered for at least 1 day.

18. The method of claim 17, wherein the daily dose mifepristone ranges from about 100 mg/day to about 2000 mg/day, or about 300 mg/day.

19. The method of claim 10, wherein the monitoring includes monitoring in an outpatient setting.
